(19) United States
(12) Patent Application Publication
(10) Pub. No.: US 2017/0209030 A1
(43) Pub. Date: Jul. 27, 2017

(54) ENDOSCOPE

(71) Applicant: OLYMPUS CORPORATION, Tokyo (JP)

(72) Inventors: Yosuke NAKAO, Tokyo (JP); Susumu AONO, Tokyo (JP)

(73) Assignee: OLYMPUS CORPORATION, Tokyo (JP)

(21) Appl. No.: 15/482,858

(22) Filed: Apr. 10, 2017

Related U.S. Application Data
(63) Continuation of application No. PCT/JP2015/075884, filed on Sep. 11, 2015.

(30) Foreign Application Priority Data
Mar. 5, 2015 (JP) 2015-043914

(51) Int. Cl. A61B 1/06; H04N 5/225; A61B 1/005; A61B 1/05; A61B 1/07

(52) U.S. Cl. CPC A61B 1/0623; A61B 1/05; A61B 1/07; A61B 1/0676; A61B 1/0055; H04N 5/2256; H04N 2005/2255

(57) ABSTRACT

An endoscope includes: an insertion portion including a distal end rigid portion, a bending portion, and an endoscope tube portion; an image pickup apparatus including an image pickup device; a first optical system having a first optical axis which is in parallel with a longitudinal axis; a second optical system including a first objective optical system having a second optical axis intersecting with the first optical axis, the second optical system being configured to bend the second optical axis to make the second optical axis coincident with the first optical axis; and a rotation driving apparatus that rotates the image pickup device, the first optical system, and the second optical system together around a rotation axis which is in parallel with the first optical axis.

### CROSS REFERENCE TO RELATED APPLICATION

This application is a continuation application of PCT/JP2015/075884 filed on Sep. 11, 2015 and claims benefit of Japanese Application No. 2015-043914 filed in Japan on Mar. 5, 2015, the entire contents of which are incorporated herein by this reference.

### BACKGROUND OF THE INVENTION

1. Field of the Invention

The present invention relates to an endoscope including a bending portion.

2. Description of the Related Art

Endoscopes are used in medical fields and industrial fields. Endoscopes used in medical fields have an elongated insertion portion to be inserted into a body, and some types of such endoscopes include a bending portion at the insertion portion.

According to an endoscope including a bending portion at an insertion portion, the bending portion is appropriately bent, thereby enabling the insertion portion to be smoothly inserted into a deep part of a body and enabling an observation optical system to be directed in a desired direction to perform observation.

For example, as shown in FIG. 1A, when performing observation in a narrow, elongated, and recessed cavity 1a with an insertion portion 2 introduced into a lumen 1, a doctor places a distal end portion 3 at a deep part in the cavity 1a, and then observes the wall of the cavity. The reference numeral 5 indicates a flexible tube portion, and the insertion portion 2 includes, in a linked manner, the distal end portion 3, a bending portion 4, and the flexible tube portion 5 in this order from the distal end side.

When the doctor observes the inner wall in the cavity 1a, the doctor performs the observation by changing the position of the distal end portion 3 shown by the solid lines in FIGS. 1B and 1C, for example, to the position shown by the solid lines, the position shown by two-dot chain lines, or the position shown by the dotted lines.

At this time, the doctor performs examination with hand operation of pulling back the insertion portion 2 or twisting the insertion portion 2, while changing an observation field of view by performing operation of bending the bending portion 4 in the up, down, right or left direction. Note that FIG. 1C illustrates the insertion portion viewed from the direction of the arrow Y1c in FIG. 1B.

For example, the endoscope disclosed in Japanese Patent Application Laid-Open Publication No. 2004-147777 includes an objective lens unit in which an objective lens is disposed, a movable frame disposed in the objective lens unit and including a field of view direction adjusting lens for adjusting a field of view direction of the objective lens, and adjusting means that transmits a rotation driving force to the movable frame to adjust the field of view direction of the objective lens, and the endoscope enables the observation to be performed by transmitting the rotation driving force to the movable frame to rotate the movable frame, and thereby adjusting the field of view direction of the field of view direction adjusting lens.

In the paragraph [0029] and FIG. 2 in the Japanese Patent Application Laid-Open Publication No. 2004-147777, it is recited that, as the thickness of the lens becomes thinner, the angle of view becomes larger and a field of view range becomes wider than a field of view range in the case where the thickness of the lens is uniform. If examination in the cavity 1a is performed with this endoscope, a wide range of region can be observed at one time. As a result, the operation time can be reduced.

### SUMMARY OF THE INVENTION

An endoscope according to an aspect of the present invention includes: an insertion portion that includes a distal end rigid portion provided at a distal end and including a longitudinal axis, a bending portion provided on a proximal end side with respect to the distal end rigid portion, and an endoscope tube portion provided on the proximal end side with respect to the bending portion; an image pickup apparatus including an image pickup device provided at the distal end rigid portion; a first optical system including a first optical axis along which light from a subject enters the image pickup device, the first optical axis being in parallel with the longitudinal axis; a second optical system provided on a distal end side with respect to the first optical system and including a first objective optical system that includes a second optical axis along which the light from the subject enters the first optical system, the second optical axis intersecting with the first optical axis, the second optical system being configured to bend the second optical axis to make the second optical axis coincident with the first optical axis; and a rotation driving apparatus disposed at the distal end rigid portion, and configured to rotationally move the image pickup device, the first optical system, and the second optical system together around a rotation axis which is in parallel with the first optical axis.

### BRIEF DESCRIPTION OF THE DRAWINGS

FIG. 1A illustrates observation to be performed by placing a distal end portion of an insertion portion in a narrow, elongated, and recessed cavity.

FIG. 1B illustrates the observation to be performed by placing the distal end portion of the insertion portion in the narrow, elongated, and recessed cavity.

FIG. 1C illustrates the observation to be performed by placing the distal end portion of the insertion portion in the narrow, elongated, and recessed cavity.

FIG. 2 shows an endoscope system including an endoscope according to a present embodiment.

FIG. 3 illustrates a distal end surface of the insertion portion.

FIG. 4 is a cross-sectional view taken along the arrow line Y4-Y4 in FIG. 3, which illustrates a configuration of the distal end portion of the insertion portion.

FIG. 5 is a cross-sectional view taken along the arrow line Y5-Y5 in FIG. 4, which illustrates a rotation driving apparatus.

FIG. 6A illustrates a working of the endoscope system according to the present embodiment and a procedure for placing the insertion portion in the vicinity of a target observation site through a trocar and then placing the distal end portion in the cavity.

FIG. 6B illustrates the working of the endo scope system according to the present embodiment and the procedure for placing the insertion portion in the vicinity of the target observation site through the trocar and then placing the distal end portion in the cavity.

FIG. 7A is an enlarged view of a part shown by the arrow Y7 in FIG. 6B, which illustrates intra-cavity observation.

FIG. 7B is an enlarged view of the part shown by the arrow Y7 in FIG. 6B, which illustrates the intra-cavity observation.

FIG. 8 illustrates another exemplary configuration of the rotation driving apparatus.

FIG. 9A illustrates an exemplary configuration of an illumination optical portion provided at a distal end rigid portion.

FIG. 9B illustrates an exemplary configuration of the illumination optical portion provided at the distal end rigid portion.

FIG. 10A illustrates a configuration in which the illumination optical portion is provided to a first objective lens frame that is rotatable with respect to the distal end rigid portion.

FIG. 10B illustrates a configuration in which the illumination optical portion is provided to the first objective lens frame that is rotatable with respect to the distal end rigid portion.

FIG. 11 illustrates another exemplary configuration of the rotation driving apparatus that rotationally moves the first objective lens frame.

FIG. 12 illustrates a stereoscopic endoscope that rotates an apparatus main body in which a lateral-view stereoscopic optical system is provided, by using the rotation driving apparatus.

FIG. 13 illustrates a configuration of the apparatus main body which is rotationally moved by the rotation driving apparatus.

FIG. 14 illustrates the observation to be performed by placing the distal end portion of the insertion portion in the narrow, elongated, and recessed cavity.

FIG. 15 illustrates the insertion portion of the endoscope.

FIG. 16A illustrates a relation between a bending angle of the bending portion and an observation range.

FIG. 16B illustrates a relation between the bending angle of the bending portion and the observation range.

FIG. 17A illustrates a protection ring provided at the bending portion.

FIG. 17B illustrates the protection ring provided at the bending portion.

FIG. 18 illustrates a working of the endoscope.

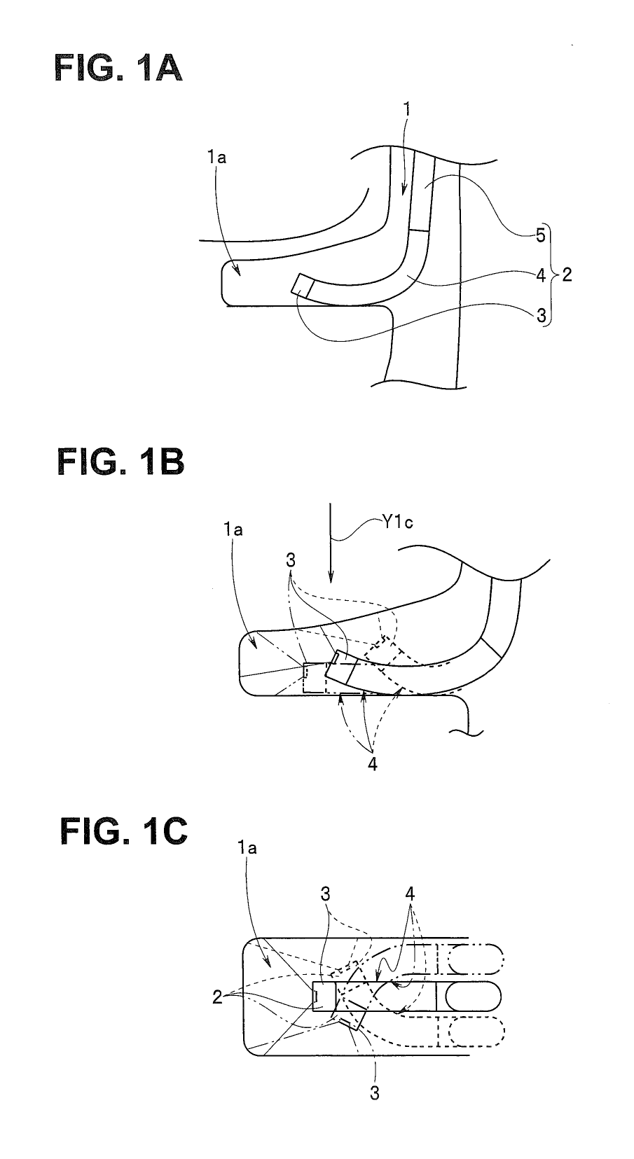

### DETAILED DESCRIPTION OF THE PREFERRED EMBODIMENTS

Hereinafter, embodiments of the present invention will be described with reference to drawings.

Note that in each of the drawings to be used in the description below, some scale sizes are made different for each of components so as to illustrate the components in recognizable sizes on the drawings. That is, the present invention is not limited only to the number, shapes, ratio of the sizes of the components, and a relative positional relation among the components shown in these drawings.

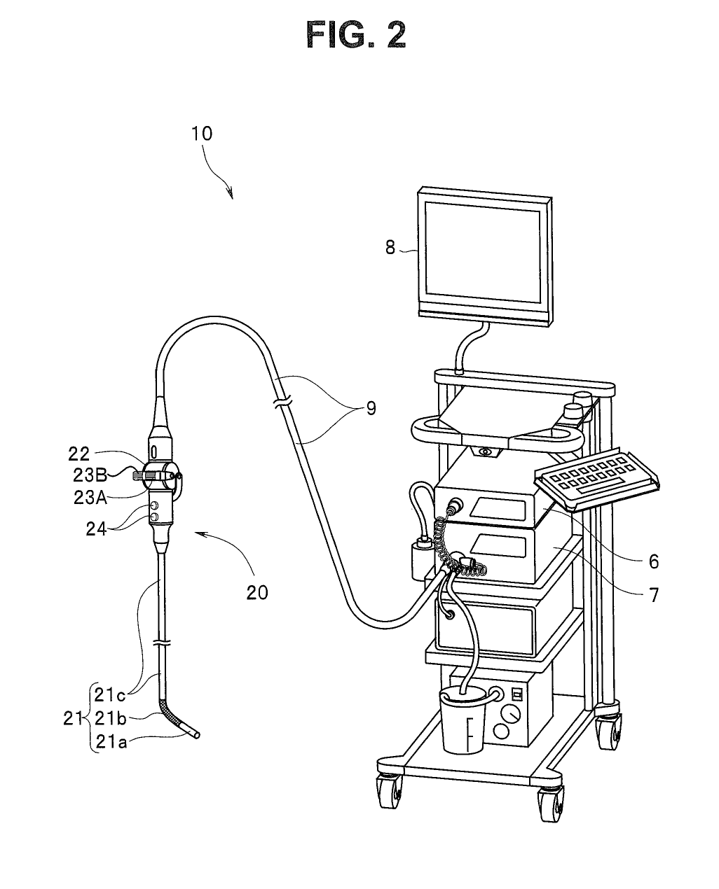

As shown in FIG. 2, an endoscope system 10 of the present invention mainly includes an endoscope 20, a video processor 6, a light source apparatus 7, and a monitor 8. In the present embodiment, the endoscope 20 is a rigid endoscope, and introduced into a body cavity through a trocar as a medical instrument.

The endoscope 20 mainly includes an elongated insertion portion 21 to be inserted into a body cavity, and an operation portion 22 provided at a proximal end with respect to the insertion portion 21. The insertion portion 21 includes in the following order from the distal end side, a distal end portion 21a, a bending portion 21b, and an endoscope tube portion 21c that are provided in a linked manner. The distal end portion 21a is provided with an image pickup device to be described later.

In the present embodiment, the endoscope tube portion 21c is a rigid tube portion. However, the endoscope tube portion 21c provided at a portion on the proximal end side with respect to the bending portion 21b is not limited to the rigid tube portion, and may have flexibility. That is, the present invention may be a flexible endoscope including what is called a flexible tube portion provided at a portion on the proximal end side with respect to the bending portion 21b.

The video processor 6 performs various kinds of signal processing, and the light source apparatus 7 supplies illumination light to the endoscope 20. The monitor 8 displays an image signal generated by the video processor 6 as an endoscopic image.

The operation portion 22 includes an up/down bending operation lever 23A and a right/left bending operation lever 23B for bending the bending portion 21b, and the bending operation levers are rotationally movable. Therefore, the bending portion 21b is bent in up and down directions and right and left directions by operating the bending operation levers 23A, 23B.

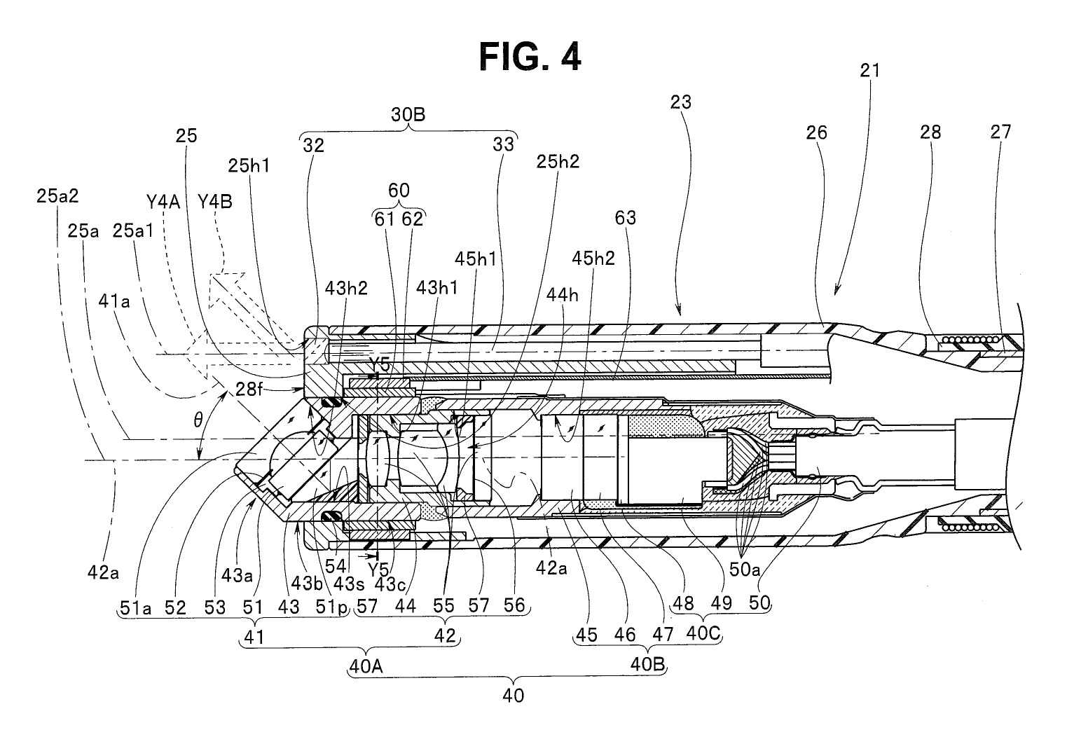

The reference numeral 24 indicates an instruction switch, and the instruction switch 24 includes a release switch that generates an instruction signal for instructing recording of a still image, a freeze switch that generates a freeze signal, or a rotation instruction switch that generates a rotation control signal for controlling the rotation driving direction by a rotation driving apparatus (see the reference numeral 60 in FIG. 4) to be described later.

The reference numeral 9 indicates a universal cord, and the universal cord is extended from the proximal end side of the operation portion 22. Signal lines to be connected to the light source apparatus 7 and the video processor 6, or an air/water feeding tube and the like are inserted in the universal cord 9.

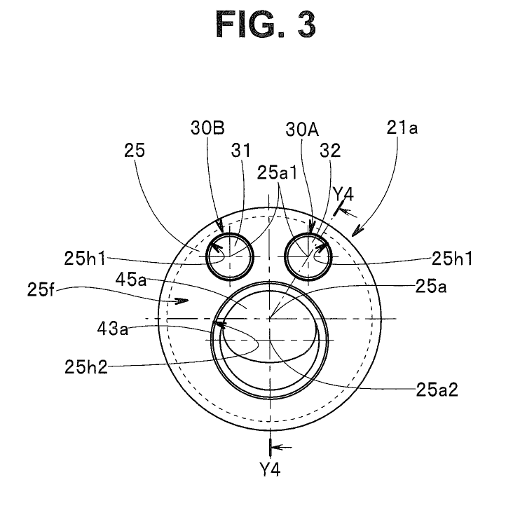

As shown in FIGS. 3, 4, the distal end portion 21a of the insertion portion 21 is provided with a distal end rigid portion 25 made of a cylindrical-shaped rigid member. The distal end rigid portion 25 is provided with an illumination optical portion 30 and an image pickup optical portion 40.

Note that the reference numeral 26 indicates a rigid ring-shaped member which is fixed on the outer circumferential surface of the distal end rigid portion 25. The reference numeral 27 indicates a distal end bending piece, and the reference numeral 28 indicates a bending rubber that covers a bending piece group including the distal end bending piece 27.

As shown in FIG. 3, a first illumination lens 31 and a second illumination lens 32 are disposed on a distal end surface 25f of the distal end rigid portion 25. The peripheral edge portion of the first illumination lens 31 and the peripheral edge portion of the second illumination lens 32 are set so as to be substantially flush with the distal end surface 25f.

The reference numeral 43a indicates a protruding portion that is a part of a first objective lens frame 43 protruding diagonally forward from the distal end surface 25f of the distal end rigid portion 25. The reference numeral 51a indicates a distal end lens which is one element of an optical lens 51, to be described later, provided on the distal end side of the first objective lens frame 43.

In the present embodiment, the first illumination lens 31 and the second illumination lens 32 are arranged in the up bending direction. The observation field of view range of the distal end lens 51a is more front side than the distal end surface 25f.

As shown in FIGS. 3 and 4, the distal end rigid portion 25 includes illumination optical portion holes 25h1 and an image pickup optical portion hole 25h2, which are through holes. The two illumination optical portion holes 25h1 are formed so as to respectively correspond to the first illumination lens 31 and the second illumination lens 32.

A central axis 25a1 of each of the illumination optical portion holes 25h1 and a central axis 25a2 of the image pickup optical portion hole 25h2 are in parallel with a longitudinal axis 25a which is a central axis of the distal end rigid portion 25.

The reference numeral 30A indicates a first illumination optical portion configured by the first illumination lens 31 and a light guide fiber 33 arranged in one of the illumination optical portion holes 25h1 and having a distal end surface that faces the proximal end surface of the illumination lens 31.

The reference numeral 30B indicates a second illumination optical portion configured by the second illumination lens 32 and a light guide fiber 33 arranged in the other of the illumination optical portion holes 25h1 and having a distal end surface that faces the proximal end surface of the illumination lens 32.

In the present embodiment, the first illumination lens 31 includes a parallel illumination axis extending forward from the distal end surface 25f along the longitudinal axis 25a. In contrast, the second illumination lens 32 includes an inclined illumination axis inclined with respect to the longitudinal axis 25a. The inclined illumination axis is gradually separated from the longitudinal axis 25a as extending forward from the distal end surface 25f.

That is, the radiation direction of the first illumination optical portion 30A is different from the radiation direction of the second illumination optical portion 30B. Illumination light is radiated from the first illumination lens 31 in the direction shown by the arrow Y4A, and illumination light is radiated from the second illumination lens 32 in the direction shown by the arrow Y4B. The illumination light radiated from each of the lenses 31, 32 expands as the illumination light advances toward the front side of the illumination axis.

Note that the first illumination lens 31 includes the parallel illumination axis and the second illumination lens 32 includes the inclined illumination axis. However, contrary to the configuration, the first illumination lens 31 may include the inclined illumination axis and the second illumination lens 32 may include the parallel illumination axis.

The image pickup optical portion 40 is disposed in the image pickup optical portion hole 25h2.

The image pickup optical portion 40 includes an objective unit 40A and an image pickup unit 40B. The image pickup unit 40B is a first optical system and has a first optical axis 42a along which the light from the subject enters the image pickup device (indicated by the reference numeral 48 to be described later). The first optical axis 42a is in parallel with the longitudinal axis 25a of the distal end rigid portion 25.

On the other hand, the objective unit 40A is a second optical system and includes a first objective lens unit 41 and a second objective lens unit 42. The second objective lens unit 42 has the first optical axis 42a.

The first objective lens unit 41 includes the first objective lens frame 43 which is a pipe member formed in a flexed shape and having a flexed through hole, and optical members such as the optical lens 51, a diaphragm 52, a spacing ring 53, and a prism 51p that are arranged in the through hole of the lens frame 43 in a predetermined state.

The first objective lens frame 43 includes in the following order from the distal end side, the protruding portion 43a, a middle portion 43b, and a rotational movement portion 43c. The rotational movement portion 43c is a rotation axis portion arranged in the image pickup optical portion hole 25h2 so as to be rotationally movable. The rotational movement portion 43c is provided with a first straight hole 43h1 that has a proximal end opening with the first optical axis 42a as a central axis.

The protruding portion 43a is provided so as to be diagonally protruded from the distal end surface 25f of the distal end rigid portion 25 with respect to the longitudinal axis 25a. The protruding portion 43a is provided with a second straight hole 43h2 which has a distal end opening, with the second optical axis 41a, which intersects with the first optical axis 42a at an angle θ (θ is an acute angle, and 45 degrees in FIG. 4), as a central axis.

The middle portion 43b has a connecting space 43s and provided between the rotational movement portion 43c and the protruding portion 43a. The connecting space 43s allows a distal end side blind hole of the first straight hole 43h1 having the proximal end opening and a proximal end side blind hole of the second straight hole 43h2 having the distal end opening to be communicated with each other. As a result, a flexed through hole in which the second optical axis 41a and the first optical axis 42a intersect with each other is provided in the first objective lens frame 43.

The first straight hole 43h1 is a lens frame arranging hole into which a distal end part of a second objective lens frame 44, to be described later, is fitted so as to be rotationally movable.

The optical lens 51 including the distal end lens 51a, the diaphragm 52, and the spacing ring 53 are arranged in the second straight hole 43h2 in a predetermined state. As a result, the first objective optical system having the second optical axis 41a is configured in the first objective lens frame 43.

The connecting space 43s is a prism disposing portion in which the prism 51p which is one element of the optical lens 51 is disposed. The prism 51p causes the second optical axis 41a to bend, to make the second optical axis 41a coincident with the first optical axis 42a.

As a result, the light entered from the distal end lens 51a into the first objective optical system and advanced along the second optical axis 41a is reflected in the prism 51p, and thereafter made to be coincident with the first optical axis 42a of the first straight hole 43h1, and then advanced toward the proximal end opening of the first straight hole 43h1.

Note that the first optical axis 42a which is the central axis of the first straight hole 43h1 is arranged in parallel with the longitudinal axis 25a in the state where the rotational movement portion 43c of the first objective lens frame 43 is arranged in the image pickup optical portion hole 25h2. In addition, the second optical axis 41a which is the central axis of the second straight hole 43h2 is arranged inclined at the angle θ with respect to the longitudinal axis 25a. That is, the first objective optical system is configured as an oblique-view optical system.

The second objective lens unit 42 includes the second objective lens frame 44, and optical members such as an optical lens 55, a diaphragm 56, a spacing ring 57, etc., that are arranged in the through hole of the lens frame 44 in a predetermined state.

The second objective lens frame 44 is a substantially straight-shaped pipe member and includes a through hole 44h. The optical lens 55, the diaphragm 56, and the spacing ring 57 are arranged in the through hole 44h in the predetermined state. As a result, the second objective optical system having the second optical axis 41a is configured in the second objective lens frame 44.

The distal end part of the second objective lens frame 44 is arranged in the first straight hole 43h1 so as to be rotationally movable. In this arrangement state, the second optical axis 41a of the second objective optical system is coincident with the central axis of the first straight hole 43h1.

Note that a distal end part of an image pickup frame 45, to be described later, constituting the image pickup unit 40B is fitted on the proximal end part of the second objective lens frame 44.

The objective unit 40A is configured by fitting the distal end part of the second objective lens frame 44 in the first straight hole 43h1 of the first objective lens frame 43, as described above. As a result, the objective unit 40A includes the first objective optical system, which is an oblique-view optical system, having the second optical axis 41a and the second objective optical system having the first optical axis 42a which is in parallel with the longitudinal axis 25a. The second optical axis 41a intersects with the first optical axis 42a at the angle θ. The rotational movement portion 43c of the first objective lens frame 43 is arranged so as to be rotationally movable around the first optical axis 42a with respect to the distal end rigid portion 25.

The image pickup unit 40B is configured by the image pickup frame 45, a cover glass 46, a protection glass 47, and an image pickup apparatus 40C. The image pickup frame 45 is a substantially straight-shaped pipe member and includes a lens frame hole 45h1 and a cover glass hole 45h2. The proximal end portion of the second objective lens frame 44 is fitted on the lens frame hole 45h1. The cover glass 46 is fixed in the glass hole 45h2.

The central axis of the lens frame hole 45h1 is same as the central axis of the glass hole 45h2. In addition, the central axis of the second objective lens frame 44 is coincident with the central axis of the lens frame hole 45h1 in the state where the lens frame hole 45h1 of the image pickup frame 45 is arranged on the proximal end portion outer circumferential surface of the second objective lens frame 44. That is, the central axis of the lens frame hole 45h1 and the central axis of the glass hole 45h2 are the second optical axis 42a.

On the proximal end surface of the cover glass 46, the distal end surface of the protection glass 47 is adhered and fixed with transparent adhesive. On the proximal end surface of the protection glass 47, an image pickup device 48 constituting the image pickup apparatus 40C is integrally fixed with transparent adhesive. In this fixed state, the image pickup surface of the image pickup device 48 is arranged so as to be orthogonal to the first optical axis 41a.

Note that CCD, C-MOS, or the like is employed as the image pickup device 48.

The image pickup apparatus 40C includes the image pickup device 48, a circuit substrate 49 on which a plurality of electronic parts (not shown) are mounted, a signal cable 50 formed by integrating a plurality of signal lines 50a connected to the circuit substrate 49, and the like.

In the present embodiment, the image pickup frame 45 is fitted on the proximal end portion of the second objective lens frame 44, and after completion of focus adjustment, the image pickup frame 45 is integrally fixed to the objective lens frame 44 with soldering or the like. Then, the image pickup frame 45 is integrally fixed, with screws or the like, at a predetermined position in the image pickup optical portion hole 25h2 formed at the distal end rigid portion 25.

Therefore, the first objective lens frame 43 is rotationally movable around the first optical axis 42a with respect to the distal end rigid portion 25 and the second objective lens frame 44.

The reference numeral 54 indicates an O-ring that retains watertightness between the outer circumferential surface of the first objective lens frame 43 and the inner circumferential surface of the image pickup optical portion hole 25h2.

In the present embodiment, the distal end rigid portion 25 is provided with a rotation driving apparatus 60 that causes the first objective lens frame 43 to rotationally move.

The rotation driving apparatus 60 is an ultrasound rotation apparatus and configured by a stator frame 61 serving as a stator, a plurality of piezoelectric elements 62, and the first objective lens frame 43 serving as a rotor. The stator frame 61 is fixed at a predetermined position in the image pickup optical portion hole 25h2.

The stator frame 61 has an operation hole 61h as a central through hole. The rotational movement portion 43c of the first objective lens frame 43 is arranged in the operation hole 61h in a predetermined fitting state. That is, the central axis of the operation hole 61h is coincident with the first optical axis 42a.

The reference numeral 63 indicates driving cables connected respectively to the piezoelectric elements 62. The driving cables 63 are extended from a driver unit (not shown) provided in the video processor 6, for example.

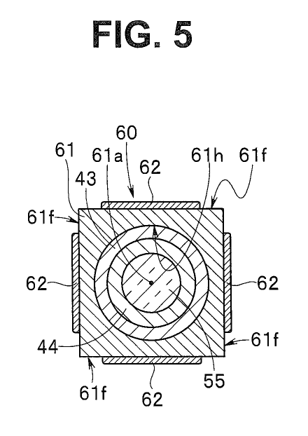

As shown in FIG. 5, the cross-sectional shape of the stator frame 61 is a square shape having at the center thereof the operation hole 61h which is a round hole. The stator frame 61 includes four outer planar faces 61f and forms a square outline.

The outer planar faces 61f are planar faces parallel to the central axis 61a of the operation hole 61h, and each of the outer planar faces 61f is provided with one of the piezoelectric elements 62, for example. The piezoelectric elements 62 are provided at positions such that the adjacent piezoelectric elements 62 are 90-degree rotation symmetrical to each other around the central axis 61a.

The four piezoelectric elements 62 are driven and controlled in response to the driving signals transmitted through the respective driving cables 63. The piezoelectric elements 62 are driven to apply oscillation to the stator frame 61.

As a result, the rotational movement portion 43c arranged in the operation hole 61h is rotated in the clockwise direction, for example, to cause the first objective lens frame 43 to rotate.

Note that it is possible to rotate the first objective lens frame 43 in the clockwise direction or counterclockwise direction by controlling the phases of the voltages to be applied to the piezoelectric elements 62.

Specifically, a doctor operates the instruction switch 24 when rotating the first objective lens frame 43. Then, a rotation control signal is outputted from the instruction switch 24 to a control section (not shown) provided in the video processor 6, for example.

The control section outputs a driving signal in the rotation direction corresponding to the rotation control signal to each of the piezoelectric elements 62. As a result, the first objective lens frame 43 is rotated in the direction desired by the doctor.

Note that the control section controls the first illumination optical portion 30A and the second illumination optical portion 30B in accordance with the rotation position of the first objective lens frame 43, in the present embodiment. That is, the control section controls a state where illumination light is radiated from the first illumination optical portion 30A, a state where illumination light is radiated from the second illumination optical portion 30B, and a state where illumination light is radiated from both of the first and second illumination optical portions 30A, 30B.

That is, the rotation amount of the first objective lens frame 43 is measured by a sensor, not shown, and the measured value is outputted from the sensor to the control section. The control section obtains the rotation position based on the measured value by a calculation section, to output a dimming control signal to the light source apparatus 7.

As a result, it is possible to prevent the flare generated by the illumination light radiated from the first illumination optical portion 30A and the illumination light radiated from the second illumination optical portion 30B jumping from the distal end lens 51a provided in the rotating first objective lens frame 43, and obtain an observation image with the best light distribution.

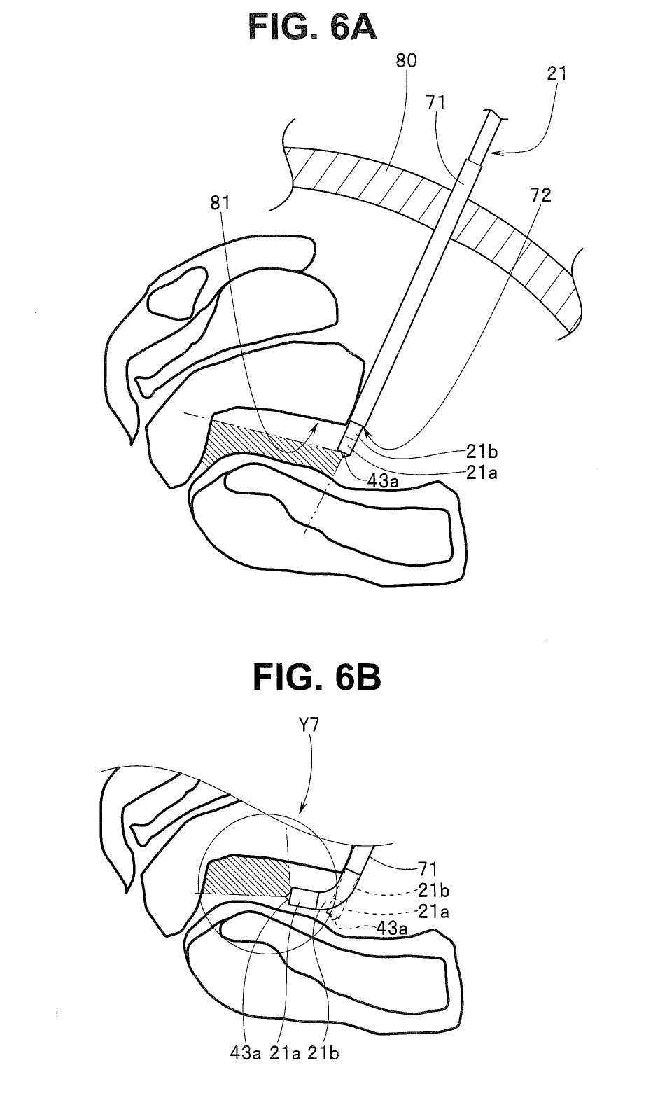

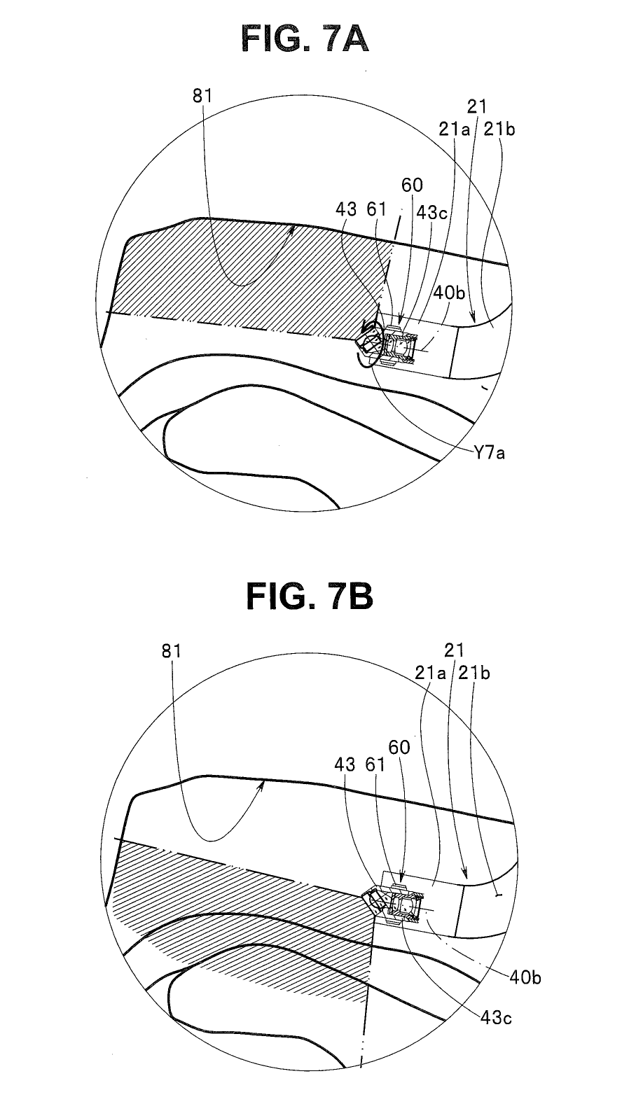

Here, the working of the endoscope system 10 will be described with reference to FIG. 6A, FIG. 6B, FIG. 7A, and FIG. 7B.

Description will be made on the case where observation is performed in the pelvic cavity inside of which is narrow and depth of which is large.

First, the doctor or the like punctures a trocar 71 into an abdomen 80, to place a distal end opening 72 of the trocar 71 in the vicinity of a pelvic cavity 81, as shown in FIG. 6A.

Next, the doctor inserts the insertion portion 21 of the endoscope 20 into the trocar 71, to lead the distal end portion 21a out of the distal end opening 72, and places the distal end portion 21a at a desired position, as shown by the dashed lines in FIG. 6B, while observing the observation field of view range in an oblique viewing direction shown by the oblique lines.

Next, the doctor operates the bending operation lever 23A to bend the bending portion 21b in the up direction. As a result, the observation field of view range changes in accordance with the bending action of the bending portion 21b, which enables the doctor to perform observation and examination of the wall on the upper surface, in the drawings, of the pelvic cavity 81.

When the doctor observes the inner wall surface of the pelvic cavity 81 over the entire circumference, for example, the doctor depresses one of the two rotation instruction switches 24 with a finger. When the doctor operates the switch that causes the first objective lens frame 43 to rotate in the counterclockwise direction, the control section that receives the rotation control signal outputs a predetermined driving signal to each of the piezoelectric elements 62 of the rotation driving apparatus 60.

Then, as shown by the arrow Y7a in FIG. 7A, the rotational movement portion 43c of the first objective lens frame 43 starts rotating in the counterclockwise direction around the first optical axis 42a. As a result, without the hand operation of twisting the insertion portion 21 or the hand operation of bending the bending portion 21b by the doctor, the observation field of view range sequentially changes in the following order from the front upper range, the front upper right range, the front right range, the front lower right range to the front lower range, as shown in FIG. 7B, in accordance with the rotation of the first objective lens frame 43.

When the doctor cancels the depression operation of the rotation instruction switch 24, the rotation of the first objective lens frame 43 is stopped. The doctor performs observation in the state where the first objective lens frame 43 is stopped.

Note that when the doctor would like to rotate the first objective lens frame 43 in the clockwise direction, the doctor has only to depress the other of the two rotation instruction switches 24, which is different from the above-described one.

As described above, the first lens frame 43 is provided so as to be rotationally movable with respect to the distal end rigid portion 25 that constitutes the distal end portion 21a of the insertion portion 21 of the endoscope 20. In addition, the first lens frame 43 is provided with the protruding portion 43a which protrudes from the distal end surface 25f of the distal end rigid portion 25 diagonally with respect to the longitudinal axis 25a and includes the oblique-view optical system

With such a configuration, after the distal end portion 21a is placed in the narrow cavity, the rotational movement portion 43c is rotated around the first optical axis 42a by the rotation driving apparatus 60, which enables the doctor to perform observation while changing the observation field of view range in accordance with the rotation of the first objective lens frame 43, without performing the hand operation of twisting the insertion portion 21 or the hand operation of bending the bending portion 21b.

Therefore, even an inexperienced doctor can surely and smoothly perform observation of the inside of the cavity after placing the distal end portion 21a of the insertion portion 21 in the narrow and deep cavity.

Note that it is possible to provide an oblique viewing endoscope having an observation field of view range desired by a doctor by appropriately setting the above-described angle θ.

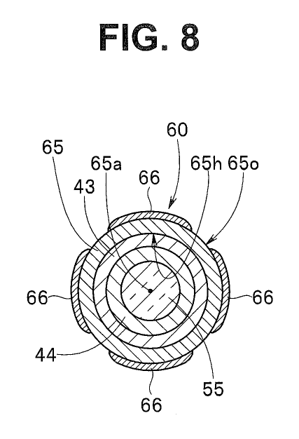

In addition, in the above-described embodiment, the cross-sectional shape of the stator frame 61 is the square shape with the round hole at the center. However, as shown in FIG. 8, the cross-sectional shape of a stator frame 65 may be an annular ring shape including at the center thereof an operation hole 65h which is a round hole.

In the configuration, for example, four piezoelectric elements 66 formed so as to be arranged on the circumferential surface are provided on the outer circumferential surface 65o of the stator frame 65 such that the adjacent piezoelectric elements 66 are 90-degree rotation symmetrical to each other around the central axis 65a.

Other configurations and working are the same as those in the above-described embodiment, and the same components are attached with the same reference numerals and description thereof will be omitted.

In addition, in the above-described embodiment, the first illumination lens 31 and the second illumination lens 32 are provided on the distal end surface 25f of the distal end rigid portion 25 and the illumination light radiation direction of the first illumination lens 31 is made different from the illumination light radiation direction of the second illumination lens 32, and then the control section performs control to cause each of the illumination optical portions 30A and 30B to radiate the illumination light in accordance with the rotation position of the first objective lens frame 43, to prevent the flare generated by the illumination light jumping from the distal end lens 51a and enable an observation image to be obtained with the best light distribution.

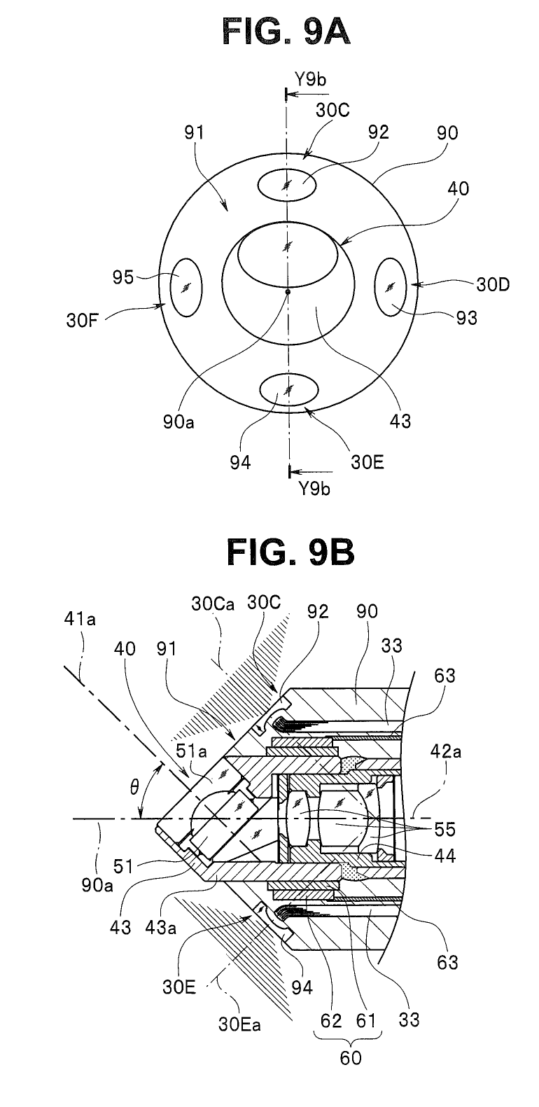

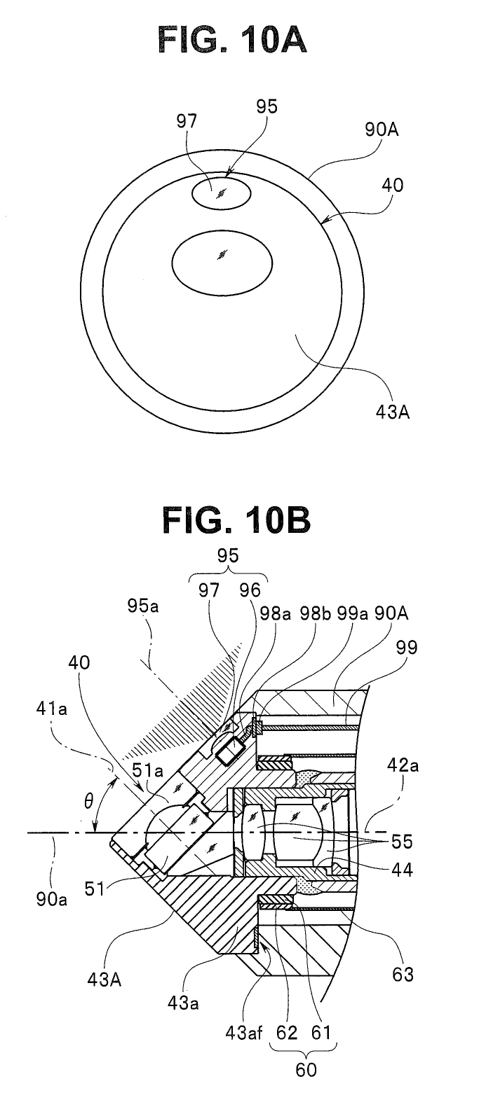

However, the illumination optical portion shown in FIG. 9A, FIG. 9B, FIG. 10A, or FIG. 10B may be provided, to prevent the flare generated by the illumination light jumping from the distal end lens 51a and enable an observation image to be obtained with the best light distribution.

As shown in FIG. 9B, a distal end rigid portion 90 in the present embodiment is formed in a cone-tapered shape. The distal end rigid portion 90 includes at the center thereof an image pickup optical portion 40 which is similar to the one in the above-described embodiment along a longitudinal axis 90a which is a central axis.

In the present embodiment, a conical inclined surface 91, which is provided so as to surround the image pickup optical portion 40 of the distal end rigid portion 90, includes four illumination optical portions 30C, 30D, 30E, and 30F, for example, as shown in FIG. 9A.

Note that the view shown in FIG. 9B is a cross-sectional view taken along the arrow line Y9b-Y9b in FIG. 9A. In addition, the reference numerals 92, 93, 94, and 95 in the drawing indicate illumination lenses, and provided corresponding to the light guide fibers 33 which respectively constitute the illumination optical portions 30C, 30D, 30E, and 30F.

In the present embodiment, the illumination lenses 92, 93, 94, and 95 are identical lenses. As shown in FIG. 9A, the illumination lenses 92, 93, 94, and 95 are provided at positions such that the adjacent illumination lenses are 90-degree rotation symmetrical to each other around the longitudinal axis 90a.

As shown in FIG. 9B, in the present embodiment, illumination axes 30Ca, 30Ea of the illumination optical portions 30C, 30E are set at the angle θ with respect to the longitudinal axis 90a such that the angle is same as the inclination angle of the second optical axis 41a of the oblique-view optical system of the image pickup optical portion 40.

Note that illumination axes 30Da, 30Fa of illumination optical portions 30D, 30F are also set at the angle θ with respect to the longitudinal axis 90a, though illustrations thereof are omitted.

The radiation range of the illumination light radiated from each of the lenses 92, 93, 94 and 95 is set as shown by the hatching in the drawing such that the best light distribution is obtained and the illumination light is prevented from jumping into the distal end lens 51a.

Other configurations are the same as those in the above-described embodiments, and the same components are attached with the same reference numerals and description thereof will be omitted.

With such a configuration, a predetermined amount of illumination light is constantly radiated from each of the illumination lenses 92, 93, 94, and 95 without the need for the control section to control whether or not to cause each of the illumination optical portions 30C, 30D, 30E, and 30F to radiate illumination light, to thereby be capable of preventing the defect such as the flare generated by the illumination light jumping from the distal end lens 51a and capable of obtaining an observation image with the best light distribution.

As shown in FIGS. 10A and 10B, a distal end rigid portion 90A of the present embodiment is also formed in a cone-tapered shape. As shown in FIG. 10B, the distal end rigid portion 90 includes at the center thereof an image pickup optical portion 40 along the longitudinal axis 90a.

In the present embodiment, the distal end lens 51a and an illumination optical portion 95 are provided on the distal end surface of the protruding portion 43a of a first objective lens frame 43A that constitutes the image pickup optical portion 40.

The illumination optical portion 95 is configured by an LED 96 as a light-emitting element and an illumination lens 97.

As shown in FIG. 10A, the illumination optical portion 95 is arranged at a position which is outer side than the outer circumference of the distal end lens 51a and includes, at the optimal position, the illumination lens 97 which is designed so as to obtain the best light distribution and prevent jumping of the illumination light into the distal end lens 51a.

The illumination axis 95a of the illumination optical portion 95 is set at the angle θ with respect to the longitudinal axis 90a such that the angle is same as the inclination angle of the second optical axis 41a of the oblique-view optical system of the image pickup optical portion 40.

The reference numerals 98a, 98b, and 99 indicate a first electrode, a second electrode, and an LED cable, respectively.

The first electrode 98a includes one end electrically connected to the LED 96 and the other end electrically connected to the second electrode 98b. The second electrode 98b is a ring electrode and formed on a proximal end surface 43af of the protruding portion 43a as an annular ring having a predetermined width dimension.

The LED cable 99 supplies power to the LED 96. The reference numeral 99a indicates a contact portion. The contact portion 99a is constantly abutted against the second electrode 98b with a biasing force of a biasing member, not shown.

Other configurations are the same as those in the above-described embodiments, and the same components are attached with the same reference numerals and description thereof will be omitted.

With such a configuration, the first objective optical system which configures the oblique-view optical system and has the second optical axis 41a, and the illumination optical portion 95 are provided at the protruding portion 43a of the first objective lens frame 43A, thereby allowing the constant light distribution of the illumination optical portion 95 to the oblique-view optical system to be maintained, irrespective of whether the first objective lens frame 43A is in a stopped state or a rotationally moving state. Therefore, such a configuration enables the defect such as the flare generated by the illumination light jumping from the distal end lens 51a to be prevented and enables an observation image to be obtained with the best light distribution.

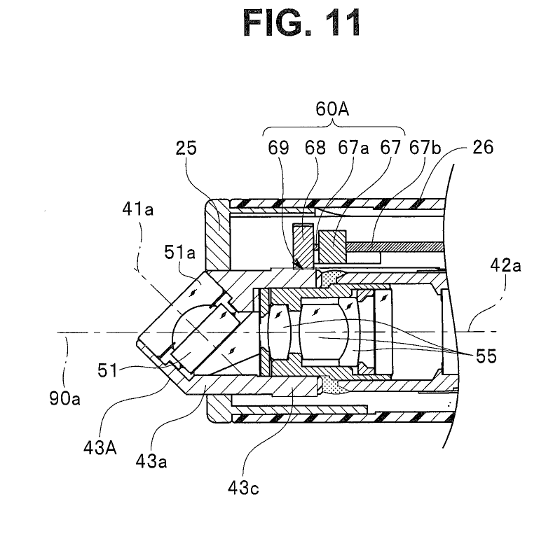

In addition, the rotation driving apparatus 60 is not limited to the ultrasound rotation apparatus configured by the stator frame 61 serving as the stator, the plurality of piezoelectric elements 62, and first objective lens frame 43 serving as the rotor, and may have the configuration shown in FIG. 11.

A rotation driving apparatus 60A shown in FIG. 11 is configured by a driving motor 67, a gear 68 provided to a motor shaft 67a, and a meshing portion 69 formed on an outer circumferential surface of the rotational movement portion 43c that configures a first objective lens frame 43B. The reference numeral 67b indicates a motor cable that supplies power to the motor 67.

The driving motor 67 is integrally fixed to a predetermined position of the distal end rigid portion 25. The motor 67 is switchable between the driving state and the stopped state based on the operation of the instruction switch 24. Other configurations are the same as those in the above-described embodiments, and the same components are attached with the same reference numerals and description thereof will be omitted.

According to the configuration, the driving motor 67 is brought into the driving state, and thereby the rotational force of the gear 68 is transmitted to the rotational movement portion 43c through the meshing portion 69, to cause the rotational movement portion 43c to be rotated around the first optical axis 42a. As a result, the same working and effects as those in the above-described embodiments can be obtained.

In addition, in the above-described embodiment, the objective unit 40A as the second optical system disposed at the distal end rigid portion 25 is configured by the first objective lens unit 41 and the second objective lens unit 42, and the first objective lens unit 41 is rotationally movable with respect to the distal end rigid portion 25 and the second objective lens unit 42.

However, the endoscope may be configured such that the image pickup device 48 and the image pickup unit 40B which is the first optical system are rotationally movable around the first optical axis 42a together with the objective unit 40A which is the second optical system.

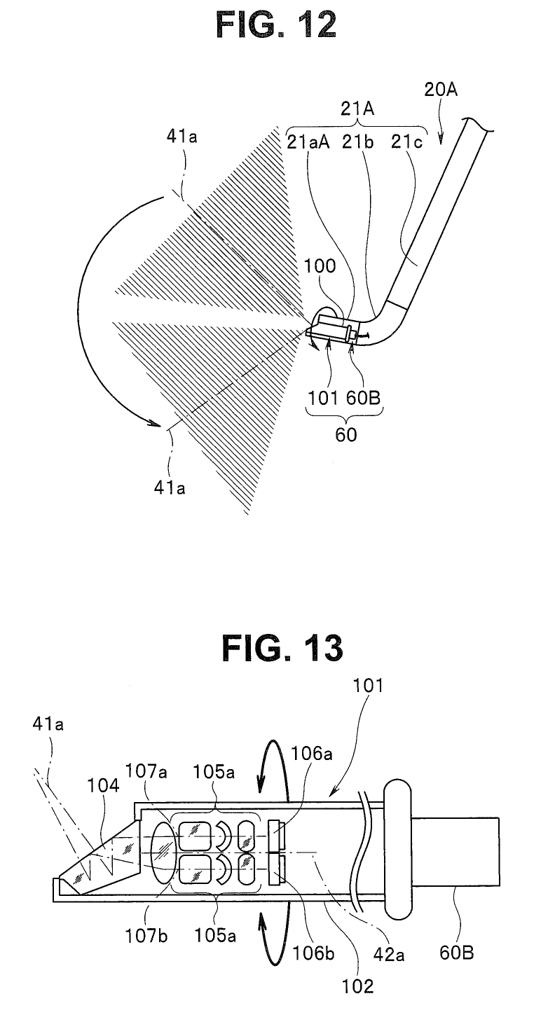

Specifically, as shown in FIGS. 12 and 13, an endoscope 20A includes an observation field of view range changing apparatus 100 in a distal end portion 21aA of an insertion portion 21A. The observation field of view range changing apparatus 100 includes an apparatus main body 101 and a rotation driving apparatus 60B.

The apparatus main body 101 includes a frame member 102, and inside the frame member 102, the second optical system having the second optical axis 41a, the first optical system having the first optical axis 42a, and the image pickup device 48 are disposed.

The rotation driving apparatus 60B is a micromotor, for example, and causes the apparatus main body 101 to rotationally move around the first optical axis 42a.

The endoscope 20A of the present embodiment is a stereoscopic endoscope and includes a stereoscopic optical system 103 in the frame member 102. The stereoscopic optical system 103 includes: a single refractive prism 104 as the second optical system, which includes a second optical axis 41a and is configured to separate light into a left light flux and a right light flux; a pair of separation image pickup optical systems 105a, 105b as the first optical system arranged in parallel behind the prism 104; and a pair of image pickup devices 106a, 106b respectively corresponding to the separation image pickup optical systems 105a, 105b. The first optical axis 42a exists at a middle position between a first optical axis 107a extending from the first separation image pickup optical system 105a to the first image pickup device 106a and a second optical axis 107b extending from the second separation image pickup optical system 105b to the second image pickup device 106b.

The image pickup devices 106a, 106b output photoelectrically converted electric signals to the video processor 6. The signals processed in the video processor 6 are converted through a scan converter (not shown) so as to be able to be displayed on a same screen as right and left stereoscopic images, and then displayed as the right and left stereoscopic images on a stereoscopic monitor 8.

An observer can capture the left and right images with the left and right eyes, respectively, through a stereoscopic glass, for example, and perform stereoscopic observation.

Note that the stereoscopic rigid endoscope 20A is capable of correcting the direction of gravitational force of the images in accordance with the rotation.

Since the observation field of view range changing apparatus 100 including the apparatus main body 101 and the rotation driving apparatus 60B is thus provided in the distal end portion 21aA of the insertion portion 21A, the same working and effects as those in the above-described embodiments can be obtained by rotating the apparatus main body 101 by the rotation driving apparatus 60B.

Incidentally, endoscopes are used in medical fields, industrial fields, etc. Endoscopes used in the medical fields include an elongated insertion portion to be inserted into a body cavity, and the insertion portion is commonly provided with a bending portion.

With the endoscopes including the bending portion at the insertion portion, the insertion portion can be smoothly inserted into a deep part of the body and observation can be performed by directing the observation optical system in a desired direction, by appropriately bending the bending portion.

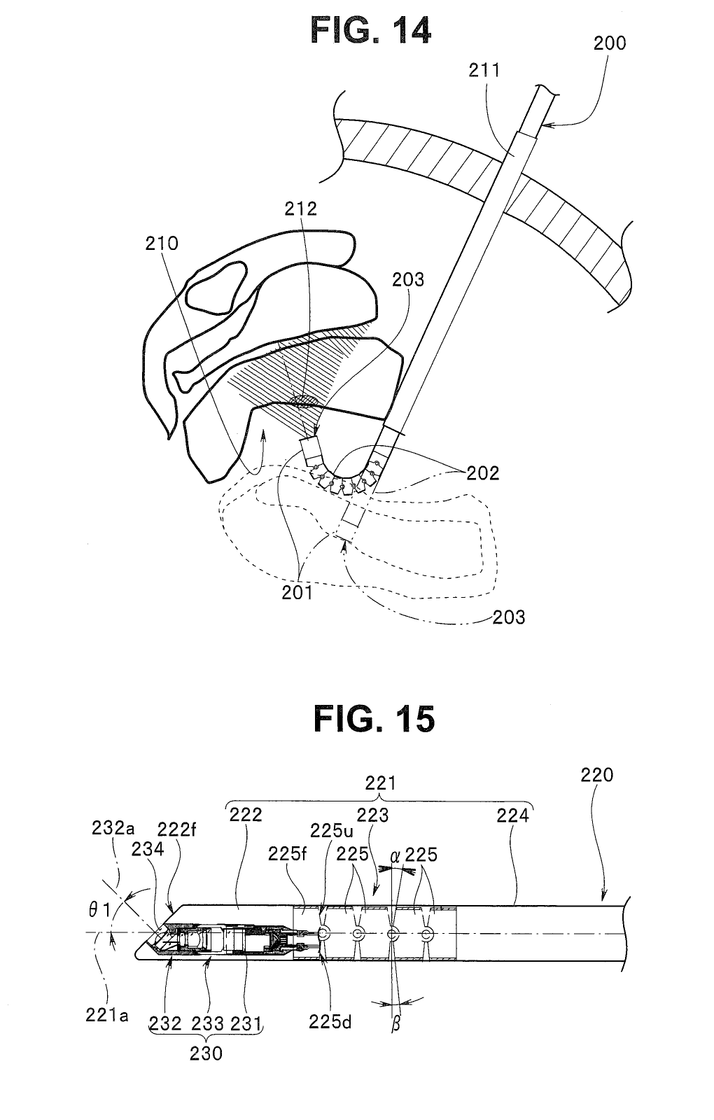

For example, when performing endoscopic observation, a doctor places a distal end portion 201 of an endoscope 200 in the vicinity of a cavity 210 through a trocar 211, as shown in FIG. 14. Then, in order to observe a site to be observed 212 in the cavity 210, the doctor causes a bending portion 202 to bend such that an observation range of an observation window (not shown) provided on a distal end surface 203 of the distal end portion 201 is set to the rear side of a longitudinal axis 204 of the insertion portion, to thereby be able to perform observation.

However, if the cavity 210 including the part shown by the dashed lines is the pelvic cavity, the pelvic cavity does not include a large space for placing the endoscope insertion portion. Therefore, after the distal end portion 201 is led out from the trocar 211 as a medical instrument, the entirety of the bending portion 202 cannot be further led out into the cavity, which has resulted in difficulty in observing the site to be observed 212 by performing bending operation.

Note that the bending portion 202 is configured by coupling eight bending pieces, for example.

In view of the above-described difficulty, there is a desire for an endo scope capable of performing wide range observation in the cavity by operating the bending portion, with the distal end portion of the insertion portion being placed in the narrow and deep cavity.

An endoscope 220 of the present embodiment is a rigid endoscope and introduced into a body cavity through a trocar. The endoscope 220 includes an elongated insertion portion 221 to be inserted into the body cavity and also includes an operation portion (not shown) at the portion on the proximal end side with respect to the insertion portion 221.

The insertion portion 221 includes, in a linked manner, a distal end portion 222, a bending portion 223, and an endoscope tube portion 224 in this order from the distal end side. The distal end portion 222 is provided with an image pickup optical portion 230 including an image pickup device 231. The image pickup optical portion 230 includes an objective unit 232 and an image pickup unit 233.

In the present embodiment, a distal end surface 222f of the distal end portion 222 is formed as an inclined surface intersecting with a longitudinal axis 221a, and a distal end lens 234 is arranged on the distal end surface 222f. The objective unit 232 including the distal end lens 234 is configured by including a plurality of optical members, not shown, such as an optical lens, a diaphragm, a spacing ring, a prism, and the like, and the objective optical system is an oblique-view optical system having an optical axis 232a intersecting with the longitudinal axis 221a of the insertion portion 221 at the angle θ1 as a first angle.

The image pickup unit 233 is configured by including a cover glass, a protection glass, which are not shown, and the image pickup device 231, etc.

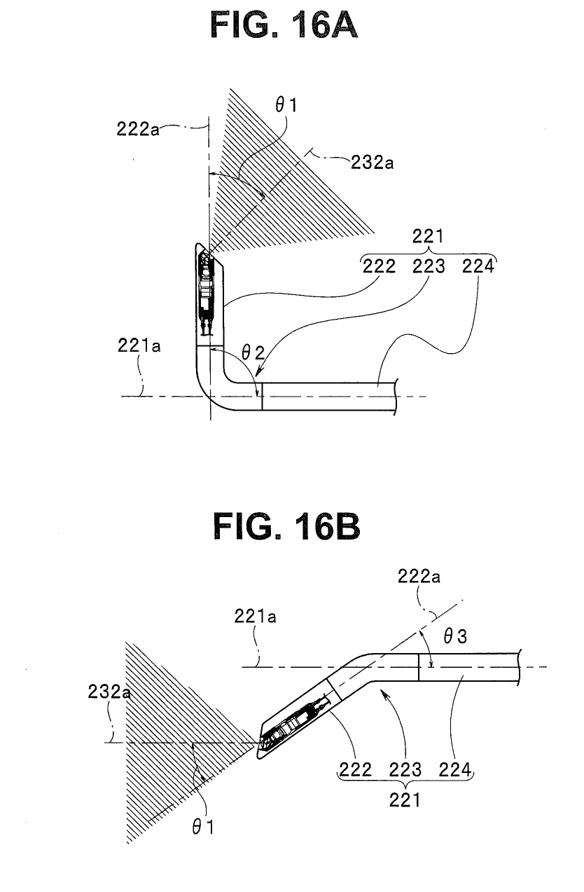

The bending portion 223 is bent in up and down directions by coupling a plurality of, for example, five bending pieces 225 in a rotationally movable manner. In the present embodiment, a direction of a distal end portion distal-most end 222a which is more distal end side than the distal end lens 234 is the down direction, and the opposite direction across the longitudinal axis 221a is the up direction. Therefore, the observation range of the oblique-view optical system, which is shown by the oblique lines (see FIGS. 16A, 16B, etc.) is set, in advance, to the forward-diagonally upper direction of the longitudinal axis 221a.

An inclination angle α of an up bending abutting surface 225u of each of the bending pieces 225 is set to be an angle different from an inclination angle (3 of a down bending abutting surface 225d of each of the bending pieces 225. In the present embodiment, since the observation range of the oblique-view optical system is set to the forward-diagonally upper direction, the inclination angle α is set to be larger than the inclination angle β.

Therefore, when the bending portion 223 is bent maximally in the direction of the observation range of the oblique-view optical system, the maximum bending angle in the direction of the observation range of the oblique-view optical system becomes larger than the maximum bending angle at the time when the bending portion 223 is bent maximally in the direction opposite to the direction of the observation range of the oblique-view optical system across the longitudinal axis 221a.

The bending portion 223 is bent in the up and down directions by operating a bending lever (not shown) provided at the operation portion.

In the present embodiment, the bending portion 223 is bent as shown in FIGS. 16A and 16B.

The bending state shown in FIG. 16A is a first bending angle state in which the bending portion 233 is bent maximally in the up direction, and a central axis 222a of a distal end portion 222 intersects with the longitudinal axis 221a of the insertion portion 221 at an angle θ2 which is a second angle. On the other hand, the bending state shown in FIG. 16B is a second bending angle state in which the bending portion 223 is bent maximally in the down direction, and the central axis 222a of the distal end portion 222 intersects with the longitudinal axis 221a of the insertion portion 221 at an angle θ3 which is a third angle.

The angle θ2 is set to be larger than the angle θ1, and the angle θ3 is set to be an angle equal to or approximate to the angle θ1.

Setting the angle θ3 as described above allows the optical axis 232a to be arranged in parallel or substantially in parallel with the longitudinal axis 221a in the down direction maximum bending state as shown in FIG. 16B, which enables the observation of the front side of the longitudinal axis 221a to be performed.

On the other hand, setting the angle θ2 as described above enables the observation of the rear side of the longitudinal axis 221a to be performed sufficiently, with the angle θ2 being 90 degrees, for example, in the up direction maximum bending state as shown in FIG. 16A, for example.

Note that the endoscope tube portion 224 is a rigid tube portion in the present embodiment. However, the endoscope tube portion 224 is not limited to the rigid portion, and the endoscope may be a flexible endoscope having flexibility, by providing what is called a flexible tube portion at the proximal end portion of the bending portion 223.

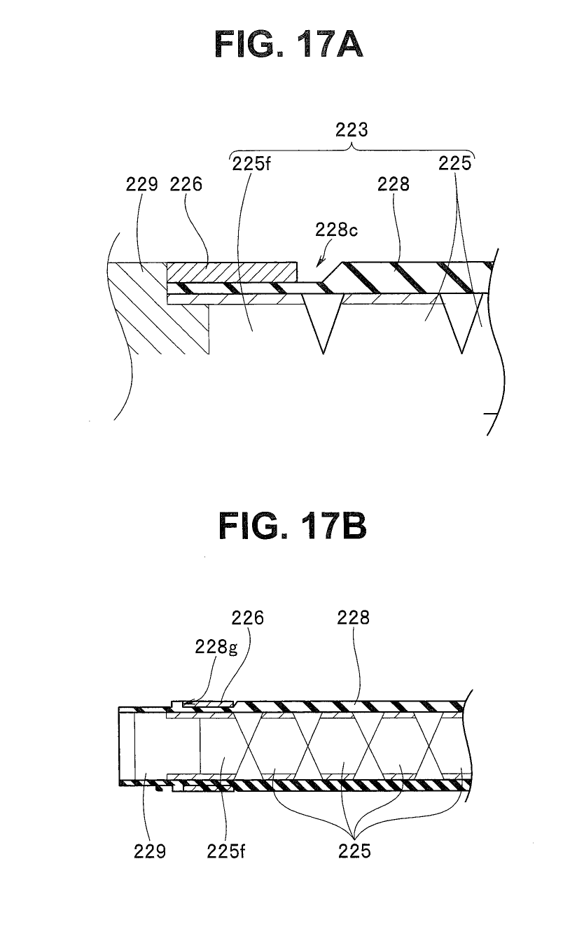

In addition, as shown in FIGS. 17A and 17B, the bending portion 223 is configured by a bending piece group 227 including a plurality of bending pieces 225 in a linked manner and a bending rubber 228 which covers the bending piece group 227. The bending rubber 228 is set to be longer than the entire length of the bending piece group 27.

The distal end side of the bending rubber 228 is arranged on the proximal end side outer circumference of a distal end rigid portion 229 provided on the distal end side with respect to a distal end bending piece 225f, and the proximal end side of the bending rubber 228 is arranged on the distal end side outer circumference of a flexible tube coupling tube (not shown) located on the proximal end side with respect to a proximal end bending piece (not shown). In addition, a protection ring 226 is disposed on each end portion of the bending rubber 228.

In the present embodiment, the protection ring 226 shown in FIG. 17A is arranged in a cutout portion 228c formed in advance at each of the end portions of the bending rubber 228. The length of the cutout portion 228c in the direction of the longitudinal axis is set in view of the width dimension of the protection ring 226, and the depth of the cutout portion 228c is set in view of the thickness of the protection ring 226 and the compression amount of the bending rubber 228.

In the endoscope 220 according to the present embodiment, the protection ring 226 is disposed in the cutout portion 228c, which allows the outer circumferential surface of the distal end rigid portion 229, the outer circumferential surface of the protection ring 226, and the outer circumferential surface of the bending rubber 228 to be flush with each other without level difference. As a result, smooth insertion of the insertion portion 221 into the trocar 211 can be achieved.

Note that a circumferential groove 228g as shown in FIG. 17B may be formed instead of forming the cutout portion 228c at each of the end portions of the bending rubber 228. The width dimension of the circumferential groove 228g is set to be equal to the width dimension of the protection ring 226.

Such a configuration not only enables the arrangement position of the protection ring 226 to be visually confirmed easily but also enables the protection ring 226 to be arranged at a predetermined position in a predetermined state.

Note that a flange part of the end surface side of the circumferential groove 228g may be cut off after the protection ring 226 is attached.

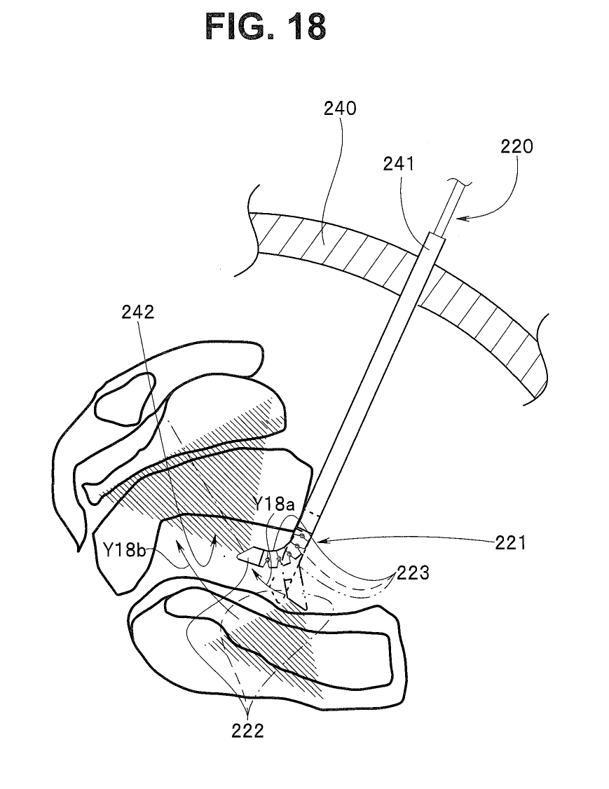

Description will be made on the working of the endoscope 220 configured as described above, with reference to FIG. 18.

When performing observation in the pelvic cavity, the doctor places the distal end portion 222 and the bending portion 223 of the insertion portion 221 in the vicinity of a pelvic cavity 242 as shown by the dashed lines through a trocar 241 punctured into an abdomen 240.

Next, the doctor operates the bending lever to bring the bending portion 223 into the down direction maximum bending state as shown by the two-dot chain lines, to recognize the position in the cavity. After that, the doctor operates the bending lever to cause the bending portion 223 to gradually bend in the up direction as shown by the arrow Y18a.

As a result, the observation range shown by the oblique lines moves as shown by the arrow Y18b in accordance with the bending action, and the doctor can observe the inside of the pelvic cavity.

Note that after placing the distal end portion 222 of the insertion portion 221 in the vicinity of the pelvic cavity 242, the doctor may perform the operation to cause the bending portion 223 to bend in the up direction to perform observation in the pelvic cavity.

Thus, the endoscope is configured by providing the objective unit 232, which is the oblique-view optical system, at the distal end portion 222 of the insertion portion 221 and providing the bending portion 223 on the proximal end side with respect to the distal end portion 222. As a result, the doctor can perform the observation of the rear side of the longitudinal axis for the inclination angle θ1 of the optical axis 232a of the oblique-view optical system by setting the maximum bending angle of the bending portion 223 to 90 degrees.

In addition, the maximum bending angle in the direction opposite to the direction of the observation range across the longitudinal axis 221a of the bending portion 223 is made substantially coincident with the angle of the oblique-view optical system, which enables the observation of the front side of the longitudinal axis 221a to be performed with the oblique-view optical system in the similar manner in the case of using a front-view endoscope.

Therefore, it is possible to provide the endoscope that is capable of placing the distal end portion 222 and the bending portion 223 in the narrow cavity by achieving a reduction in the bending portion length of the bending portion 223, to enable wide range of endoscopic observation.

### [Note]

### [Note 1] an Endoscope Including:

an insertion portion to be inserted through a medical instrument punctured into an abdominal cavity;

a distal end portion provided with an objective optical system having an optical axis that intersects with a longitudinal axis of the insertion portion at a predetermined first angle; and

a bending portion provided on a proximal end side with respect to the distal end portion and configured by coupling a plurality of bending pieces so as to be rotationally movable, wherein a maximum bending angle of the bending portion in a direction of an observation range of the objective optical system is set to be larger than a maximum bending angle of the bending portion in a direction opposite to the direction of the observation range of the objective optical system across the longitudinal axis.

### [Note 2]

The endoscope according to Note 1, wherein the maximum bending angle of the bending portion in the direction opposite to the direction of the observation range of the objective optical system across the longitudinal axis is same as the first angle or an angle approximate to the first angle.

Note that the present invention is not limited only to the above-described embodiments, and various modifications are possible without departing from the gist of the invention.

The present invention is capable of providing an endoscope that enables an observation in a narrow, elongated, and recessed cavity to be performed without a complicated hand operation.

What is claimed is:

1. An endoscope comprising:
an insertion portion that includes a distal end rigid portion provided at a distal end and including a longitudinal axis, a bending portion provided on a proximal end side with respect to the distal end rigid portion, and an endoscope tube portion provided on the proximal end side with respect to the bending portion;
an image pickup apparatus including an image pickup device provided at the distal end rigid portion;
a first optical system including a first optical axis along which light from a subject enters the image pickup device, the first optical axis being in parallel with the longitudinal axis;
a second optical system provided on a distal end side with respect to the first optical system and including a first objective optical system that includes a second optical axis along which the light from the subject enters the first optical system, the second optical axis intersecting with the first optical axis, the second optical system being configured to bend the second optical axis to make the second optical axis coincident with the first optical axis; and
a rotation driving apparatus disposed at the distal end rigid portion, and configured to rotationally move the image pickup device, the first optical system, and the second optical system together around a rotation axis which is in parallel with the first optical axis.

2. The endoscope according to claim 1, wherein
the first optical system includes a pair of separation image pickup optical systems,
the second optical system separates the light from the subject into two light fluxes, to cause one of the light fluxes to enter one of the pair of the separation image pickup optical systems and cause another of the light fluxes to enter another of the pair of the separation image pickup optical systems, and
the rotation axis is disposed at a middle position between an optical axis of one of the pair of the separation image pickup optical systems and an optical axis of the other of the pair of the separation image pickup optical systems.

3. The endoscope according to claim 2, further comprising a frame member in which the first optical system and the second optical system are disposed, wherein the frame member is rotationally movable by the rotation driving apparatus.

4. The endoscope according to claim 3, further comprising a plurality of illumination optical portions at positions which are on a distal end surface of the distal end rigid portion and which are around the frame member that is rotationally movable with respect to the distal end rigid portion.

5. The endoscope according to claim 1, wherein the endoscope tube portion is a rigid pipe.
